(19) United States
(12) Patent Application Publication
(10) Pub. No.: US 2004/0072222 A1
(43) Pub. Date: Apr. 15, 2004

(54) Peptide analogues of myelin basic protein epitopes in the treatment of experimental autoimmune encephalomyelitis (eae) and multiple sclerosis (ms)

(76) Inventors: John Matsoukas, Patra Rio (GR); Vasso Apostolopoulos, Heidelberg, Victoria (AU); Theodore Tselios, Patra Rio (GR)

Correspondence Address: John Matsoukas, Dept of Chemistry, University of Patras, Patras Rio, 26500, GR

(21) Appl. No.: 10/467,680

(22) PCT Filed: Mar. 23, 2001
(86) PCT No.: PCT/GR01/00014

(51) Int. Cl. C12Q 1/68

(52) U.S. Cl. 435/6

(57) ABSTRACT

This invention relates to novel linear and cyclic peptide analogues of Myclin Basic Protein epitopes (from guinea pig MBP₇₂₋₈₅ and human MBP₈₇₋₉₉) and their conjugates with mannan and/or KLH useful in the treatment of Experimental Autoimmune Encephalomyclitis (EAE) and Multiple Sclerosis (MS). For the first time cyclic analogues of MBP epitopes have been synthesized and shown to prevent the development of EAE. There is gathering evidence that analogues of disease-associated epitopes can be conjugated to mannan and/or KLH and actively generate antigen specific regulatory CD4/CD8 T cells and Th1/Th2 cytokines.

### TECHNICAL FIELD OF INVENTION

This invention relates to novel linear and cyclic peptide analogues of Myelin Basic Protein epitopes and their conjugates with mannan and/or KLH useful in the treatment of Experimental Autoimmune Encephalomyelitis (EAE) and Multiple Sclerosis (MS).

### INTRODUCTION BACKGROUND OF INVENTION

This invention discloses analogues of Myelin antigens and mannan/KLH conjugates for treatment of Multiple Sclerosis.

Multiple Sclerosis and Experimental Autoimmune Encephalomyelitis/Animal Model

Multiple sclerosis (MS) is a chronic disease of the central nervous system (CNS) characterized by local T cell and macrophage infiltrates, demyelination and loss of neurologic function [Steinman, 1996, Martin, et al 1992]. MS is believed to be an autoimmune disease triggered by CNS-specific CD4 T lymphocytes. Candidate autoantigens include constituents of the myelin sheath, such as myelin basic protein (MBP) and proteolipid protein (PLP). Modem approaches towards the therapeutic management of MS involve the design and use of peptide analogues of disease-associated myelin epitopes to induce peripheral T cell tolerance [Hafler, et al 1995, Hohfeld, 1997, Ota, et al 1990]. Experimental Autoimmune Encephalomyelitis (EAE), one of the best studied experimental animal models of MS, is a useful in vivo system for the evaluation of such therapeutic approaches.

Inhibition of Disease Using Guinea Pig MBP Epitope 7285 Analogues

In Lewis rats immunized with guinea pig MBP protein, encephalitogenic T cells recognizing the MBP72-85 epitope, dominated the immune response [Wauben, et al 1992]. In particular, the linear analogue Gln-Lys-Ser-Gln-Arg-Ser-Gln-Asp-Glu-Asn-Pro-Val (MBP₇₂₋₈₅) has been found to induce EAE, while substitution of the Asp residue at position 81 with Ala resulted in an analogue Gln-Lys-Ser-Gln-Arg-Ser-Gln-Ala⁸¹-GluAsn-Pro-Val (Ala⁸¹MBP₇₂₋₈₅) that prevented the induction of EAE by its parent peptide. Peptides participating in the trimolecular complex, MHC-peptide-TCR and cause an antagonist effect (ie. loss of T-cel activation) has a loss of H-bond contacts of the peptide side chains to the CDR3 loops of the TCR. In such an event this loss of H-bond contact causes an agonist peptide to become an antagonist peptide [[)egano, et al 2000]. Peptide therapy, however, is hindered due to the sensitivity of peptides to proteolytic enzymes. Continuous infusions of prohibitive amounts of peptides are necessary to elicit the necessary biological response [Matsoukas, et al 1994, Matsoukas, et al 1996]. To address the need for more stable molecules, cyclic analogues that could maintain or suppress the biological function of the original peptide, yet could also elicit a response in pharmacological quantities, were designed, synthesized and evaluated for their activity in the EAE system as well as in human peripheral blood T cells [Tselios, et al 1998, Tselios, et al 1999]. Design of potent cyclic analogues was based on Nuclear Magnetic Resonance and Molecular Dynamics studies carried out in the agonist and antagonist linear analogues, MBP₇₂₋₈₅ and Ala⁸¹MBP₇₂₋₈₅ respectively. These studies revealed a head-to-tail intramolecular proximity (ROE connectivity between βVal¹²-γGln¹ in MBP₇₂₋₈₅ and βPro¹¹H-γGln¹ in Ala⁸¹MBP₇₂₋₈₅), suggesting cyclic conformations for the two linear analogues in solution. These results led to the synthesis of the cyclic analogues: Gln¹-Lys²-Ser³-Gln⁴-Arg⁵-Ser⁶-Gln⁷-Asp⁸-Glu⁹-Asn¹⁰-Pro¹¹-Val¹²-NH₂ (c-MBP₇₂₋₈₅) Gln¹-Lys²-Ser³-Gln⁴-Arg⁵-Ser⁶-Gln⁷-Ala⁸-Glu⁹-Asn¹⁰-Pro¹¹-Val¹²-NH₂ (c-Ala⁸¹MBP₇₂₋₈₅) by cornecting the e amino group of Lys and y carboxyl group of Glu at positions 2 and 9 [Tselios, et al 1999]. Cyclization is known to restrict the number of possible conformations, allowing the possibility to diminish the unfavored conformations for approaching the receptor site in a direct peptide-receptor interaction. The c-MBP₇₂₋₈₅ analogue has comparable potency to MBP₇₂₋₈₅ in inducing EAE in Lewis rats while the clinical and histopathological manifestations of disease induced by c-MBP₇₂₋₈₅ are prevented by a linear antagonist (Ala⁸¹MBP₇₂₋₈₅) (Tselios, et al 1999]. In our studies the encephalitogenic activity of MBP₇₂₋₈₅ was completely prevented by the co-injection with c-Ala⁸¹MBP₇₂₋₈₅. Furthermore, the cyclic analogues (c-MBP₇₂₋₈₅ and c-Ala⁸¹MBP₇₂₋₈₅) were assessed for their biological activities in human peripheral blood T cells and their activity was comparable with that of linear agonist and antagonist analogues MBP₇₂₋₈₅ and Ala⁸¹MBP₇₂₋₈₅. The linear and cyclic forms of the agonist peptide MBP₇₂₋₈₅ had the comparable effect on human T cell activation and proliferation and their effect was completely reversed by co-culturing of the cells with the linear or cyclic analogues of the antagonist peptide Ala⁸¹MBP₇₂₋₈₅. The comparable potencies of linear and cyclic analogues MBP₇₂₋₈₅ and c-MBP₇₂₋₈₅ as well as of analogues Ala⁸¹MBP₇₂₋₈₅ and c-Ala⁸¹MBP₇₂₋₈₅, indicate that a cyclic conformation of the MBP₇₂₋₈₅ epitope together with a carboxyl group at position 81 is important for the function of the trirnolecular complex, MHC-peptide-T cell receptor and for the activation of EAE specified T-cells.

Demyelination and inflammation in the spinal cord at day 15 after immunization with MBP₇₂₋₈₅, is completely prevented by the co-immunization of the c-Ala⁸¹MBP₇₂₋₈₅. Spinal cord sections of an MBP₇₂₋₈₅-immunized rat showing multiple perivascular infiltrates (densely stained by Nuclear Fast Red), and patchy demyelination (oss of continuity of Luxol Fast Blue staining). In contrast, sections from a rat co-immunized with MBP₇₂₋₈₅ and c-Ala⁸¹MBP₇₂₋₈₅ shows a complete absence of inflammatory infiltrates and normal myelin structure. Cyclic analogues offer multiple advantages compared to linear counterparts, in terms of stability, duration of action and receptor selectivity.

Inhibition of Disease Using Human MBP Epitope 87-99 Analogues

Structure-Activity studies based on the human MBP₈₇₋₉₉ [Vergeli, et al 1996, Brocke, et al 1996] epitope (Val-His-Phe-Phe-Lys-Asn-Ile-Val-Thr-Pro-Arg-Thr-Pro) resulted in linear and cyclic analogues namely [Arg⁹¹,Ala⁹⁶]MBP₈₇₋₉₉ (Val-His-Phe-Phe-Arg⁹¹-Asn-Ile-Val-Thr-Ala⁹⁶-Arg-Thr-Pro) and cyclo-(87, 99)[Arg⁹¹,Ala⁹⁶]MBP87-99 (Val⁸⁷-His-Phe-Phe-Arg⁹¹-Asn-Ile-Val-Thr-Ala⁹⁶-Arg-Thr-Pro⁹⁹) which completely prevented the induction of EAE when co-injected to Lewis rats together with encephalitogenic agonist MBP₇₂₋₈₅. Blockade of MBP₇₂₋₈₅ induced EAE by the previous unrelated peptides could indicate that the mechanism of inhibition is not due to binding competition but rather due to the delivery of a negative signal by the antagonist which overcomes the agonist response possibly through the activation of antigen specific regulatory T cells.

Inhibition of Disease Through Control of Cytokine Secretion Using Mannan Conjugates of MBP Epitopes

There is gathering evidence that analogues of disease-associated epitopes can be conjugated to mannan and/or KLH and actively generate antigen specific regulatory CD4/CD8 T cells and Th1/Th2 cytokines. The ability to alter the cytokine secretion of autoreactive T cell lines through peptide or mimetic treatment even in longstanding autoimmune disease indicates that cytokine therapy might have therapeutic benefits by switching the function of myelin reactive T cells such that they are non-pathogenic. Mannan conjugates of Myelin antigens in the oxidized or reduced form (with the participation or not of KLH) are suitable tools to control cytokine secretion.

Current Peptide Therapies for MS

Current peptide therapies of multiple sclerosis include treatment with Interferon's (Interferon beta-1α and Interferon beta-1β) and glatiramer acetate (copolymer-1) which is a synthetic protein comprised of the major aminoacids Glu, Gln, Lys, Arg of MBP. These immunomodulators have been approved by the FDA for patients with relapsing-remitting MS. Interferon's given by S.C. injections, reduce the frequency, severity and duration of exacerbation but their impact on preventing disability over the long-term is not yet established. Side effects also are common and consist of reactions at the injection site, fever, myalgia and blu-like syndrome. So far the reported benefits from the use of interferon's and copolymer are marginal and therefore the need for improved therapeutics are imperative. Another approach under clinical investigation for autoimmnune suppression is the oral administration of autoantigens. In this study, orally administered antigens suppress autoimmunity in animal models, including EAE, collagen and adjuvant-induced arritis, uveitis and diabetes in the non-obese diabetic mouse. Low doses of oral antigen induce antigen-specific regulatory T-cells which act by releasing inhibitory cytokines such as transforming growth factor-beta, interleukin-4, and interleukin-10 at the target organ. Thus, one can suppress inflammation at a target organ by orally administering an antigen derived from the side of inflammation, even if it is not the target of the autoimmune response. Initial human trials of orally administered antigen have shown positive findings in patients with MS and Rheumatoid Arthritis. A double-blind, placebo-controlled, phase HI multi-center trial of oral myelin in relapsing-remitting multiple sclerosis patients is in progress, as are phase II clinical trials investigating the oral administration of type II collagen in rheumatoid arthritis, S-antigen in uveitis and insulin in type I diabetes. This promising method has the oral administration advantage over the previous methods using interferons and copolymer-1. However, issues related to the peptide nature and cost of administered substance renders the non-peptide mimetic approach, even in its infancy, an attractive goal to pursue.

Strategies in the Immunotherapy of Multiple Sclerosis and Clinical Perspectives

In the immunotherapeutic approach towards the development of therapeutic vaccines for MS the assumption is that MBP epitopes or their analogues can actively inhibit or prevent disease through the activation of antigen-specific regulatory T cells, or antibodies related to myelin sheath destruction. The myelin sheath is constituted from the proteins: MBP, Proteolipid Protein (PLP), Myelin Oligodentrocyte Glycoprotein (MOG) and heat-shock protein which are implicated in MS. Thus, epitopes of these Myelin sheath proteins are targets for irmnunotherapeutic techniques. In areas of inflammation in MS, antibodies against the minor protein MOG have been demonstrated. MOG antibodies were related to significant myelin disruption, probably by coating the myelin so that macrophages could engulf and destroy coated sections of myelin, blocking nerve impulses temporarily or permanently. Thus, we now know that antibodies do play a role in MS, and cooperate with antigen presenting cells in myelin destruction. Blocking the effects of these MOG antibodies with secondary antibodies or non-peptide mimetics might be an important avenue for future therapy.

Another direction in the immunotherapy of autoimmune diseases is the use of Multiple-Antigen Peptide (MAP) systems introduced by Tam [Tam 1990]. This system represents a novel approach to anti-peptide antibody production. It is build on a resin which bears a core of radial branching lysine dendrites on which a number of copies of a given peptide antigen can be synthesized. Lysine derivatives have been used for the solid phase synthesis of lysine cores suitable for the assembly of antigenic peptides. These peptides have found application in raising antibodies and in the preparation of synthetic vaccines. On a lysine core several different epitopes of a protein or of different proteins can be assembled to create the required antigenic synthetic protein. Following assembly of the peptide on the MAP core, the peptide is deprotected and cleaved from the support using standard techniques, yielding a highly immunogenic macromolecular structure without the need for conjugation to a carrier protein. The MAP approach has been shown to yield higher antibody titres than using monomeric peptide-conjugates. Alternatively, the two epitopes can be synthesized on alternate branches of the lysine core, using Boc and Fmoc chemistry. T-cell and B-cell epitopes can also be combined sequentially within a single linear sequence.

Another challenging strategy in the immunotherapy of MS or other autoimmune diseases is the use of peptide poly-lysine analogues conjugated with mannan via KLH in its oxidized or reduced form to develop Th1/Th2 responses followed by release of appropriate cytokines [Apostolopoulos, et al 1995, Apostolopoulos, et al 1996, Apostolopoulos, et al 1998, Lofthouse, et al 1997]. The aim of this approach is the development of a therapeutic vaccine for prevention or control of disease. Presently, antigen peptide of MBP, PLP and MOG proteins conjugated to mannan (with or without KLH) in its oxidized and reduced form are under investigation. Assays to study their effect on cellular proliferation, antibody production and cytokines secretion being determined in Lewis rats and in human peripheral blood T-cells. These studies include the development of recently rationally designed constrained cyclic antagonist peptide analogues based on MBP epitopes 72-85 and 87-99 which suppresses the development of clinical E.A.E., CNS inflammation and demyelination. The use of oxidized or reduced mannan to develop a Th1 or Th2 response (and the appropriate cytokines) to Myelin peptides expressed in MS constitutes a novel strategy for the treatrnent of disease. Mannan has been investigated extensively for its ability to generate responses in several model systems. Its adjuvant function has been shown to stem, from its ability to target the mannose receptor an antigen presenting cells. Of particular interest, mannan conjugated to peptide under oxidizing conditions induces Th1-type immune responses, while peptides conjugated to mannan under reducing conditions generate Th2-type immune responses. Antibodies, CTL, tumor protection or the secretion of IL-1, IL-2, IL-4, IL-5, IL-6, IL-7, IL-10, IL-12, IL-13, IL-15, TNF-alpha, IFN-gamma, GM-CSF, TGF-beta, cytokines have been measured by mRNA or in vitro culture of ATP/T-cells, after immunization with oxidized or reduced mannan-peptide conjugates.

### REFERENCES

Apostolopoulos, V.; Lofthouse, S.; Popovski, V.; Chelvanayagam G.; Sandrin, M.; McKenzie I. Nature Biot. 1998, 16, 278.

Apostolopoulos, V.; Pietersz, A.; McKenzie, I. Vaccine, 1996, 14, 930.

Apostolopoulos, V.; Pietersz, A.; Loveland, B.; Sandrin, M.; McKenzie, I. Proc. Natl. Acad. Science, 1995, 92, 10128.

Berezhkovskiy, L.; Pham, S.; Reich, P.; Deshpande, S. J. Pept. Res. 1999, 54, 112.

Brocke, S.; Gijbels, K.; Allegretta, M.; Ferber, I.; Piercy, C.; Blankenstein, T.; Martin, R.; Utz, U.; Karin, N.; Mitchell, D.; Veroma, T.; Waisman, A.; Gaur, A.; Conlon, P.; Ling, N.; Fairchild, P.; Wraith, D.; O'Carra, A.; Farhman, G.; Steinman, L. Nature 1996, 379, 343.

Bruggle, F.; Timmermans, M.; Van Unen, A.; Ten Hove, J.; Van De Werken, G.; Poolman, T.; Hoogerhout, P. J. Pept. Sci. 1999, 5, 381.

Chaloin, L.; Mery, J.; Van Mau, N.; Divita, G.; Heitz, F. J. Pept. Sci. 1999, 5, 381.

Degano, M.; Garcia, K.; Apostolopoulos, V.; Rudolph, M.; Teyton, L.; Wilson, I. Immunity, 2000, 12, 251.

Hafler, D.; Weiner, H. Immunol. Rev,. 1995, 144, 75.

Hohfeld, R. Brain. 1997, 120, 865.

Iwai, H.; Pluckthun, A. FEBS Let. 1999, 459, 166.

Kromboritis, E., Hatridakis, G., Barlos, K. J. Biol. Chem., 1998, 273, 10874

Kurtzke, F. Neurology, 1983, 33, 1442.

Lofihouse, S.; Apostolopoulos, V.; Pietersz, G.; Wenjun, L.; McKenzie, I. Vaccine, 1997, 15, 14, 1586.

Martin, R.; McFarland, H.; McFarlin, D. Ann. Rev. Immunol. 1992, 10, 153

Matsoukas, J.; Hondrelis, J.; Agelis, G.; Barlos, K.; Gatos, D.; Ganter, R.; Moore, D.; Moore, G. J. Med. Chem. 1994, 37, 2958.

Matsoukas, J.; Panagiotopoulos, D.; Keramida, M.; Mavromoustakos T.; Yamdagni, R.; Wu, Q.; Moore, G.; Saifeddine, M.; Hollenberg M. J. Med. Chem. 1996, 39, 3585.

Mezo, G.; Majer, Z.; valero, L.; Andreu, D.; Hudecz, F. J. Pept. Sci. 1999, 5, 272.

Oligino, L.; Lung, T.; Sastry, L.; Bigelow, J.; Cao, T.; Curran, M.; Burke, R.; Wang, S.; Krag, D.; Roller, P.; King, R. J. Biol. Chem. 1997, 272, 29046.

Ota, K; Matsui, M.; Milford, E.; Mackin, G.; Weiner, H.; Hafler, D. Nature (Lond) 1990, 346, 183.

Scott, P.; Abel-Santos, E.; Wall, M.; Wahnon, C.; Benkovic, J. Proc. Natl. Acad. Sci. USA 1999, 16, 16.

Steinman, L. Cell 1996, 85, 299.

Tam, P.; Clavijo, P.; Lu, Y.; Nussenzweig, S.; Nussenzweig, S.; Zavala, F. J. Exp. Med 1990, 171, 299.

Tamilarasu, N.; Huq, I.; Rana, T. Bioor. Med. Chem. Let. 2000, 10, 971.

Tselios, T.; Probert, L.; Kollias, G.; Matsoukas, E.; Roumelioti, P.; Alexopoulos, K.; Moore, G.; Matsoukas J. Amino Acids 1998, 14, 333.

Tselios, T.; Probert, L.; Daliani, I.; Matsoukas, E.; Troganis, A.; Gerothanassis, P.; Mavromoustakos, T.; Moore, G.; Matsoukas J. J. Med. Chem. 1999, 42, 1170.

Vergeli, M.; Hemnmer, B.; Utz, U.; Vogt, A.; Kalbus, M.; Tranquill, L.; Conlon, P.; Ling, N.; Steinman, L.; McFarland, H.; Martin R. Eur. J. Immunol. 1996, 26, 2624.

Wauben, M.; Boog, C.; Zee, R.; Joosten, I.; Scihlief, A.; Eden, W. J Exp. Med. 1992, 176, 667.

### STATE OF ART OF INVENTION

A. Conformational Models

A1. Conformational Model of Guinea Pig MBP7285

A Cyclic conformation and the role of Asp/Arg interaction in triggering activity, is disclosed in this application.

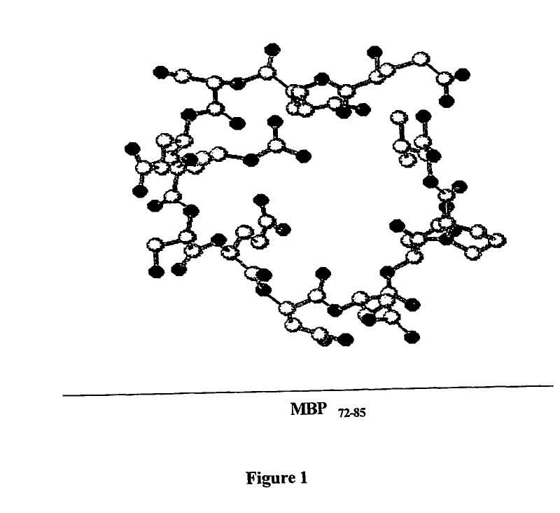

To address the need for more stable molecules, cyclic analogues which could maintain the biological function of the original peptide, yet could be able to elicit a response in pharmacological quantities, were designed, synthesised and evaluated for activity in the EAE. system. Design of the cyclic analogues of guinea pig MBP epitope 72-85 was based on Nuclear Magnetic Resonance and Molecular Dynamics studies carried out in the Guinea Pig agonist linear MBP₇₂₋₈₅ epitope. These studies revealed a head to tail intramolecular proximity (ROE connectivity between βVal12-γGln1) suggesting a cyclic conformation for MBP₇₂₋₈₅ (FIG. 1). We therefore, synthesised the cyclic analogues Gln¹-Lys²-Ser³-Gln⁴-Arg⁵-Ser⁶-Gln⁷-Asp⁸-Glu⁹-Asn¹⁰-Pro¹¹-Val¹²-NH₂ (1) and Gln¹-Lys²-Ser³-Gln⁴-Arg⁵-Ser⁶-Gln⁷-Ala⁸-Glu⁹-Asn¹⁰-Pro¹¹-Val¹²-NH₂ (2) by connecting the ε amino group of Lys and γ carboxyl group of Glu at positions 2 and 9. The cyclic analogue 1 was assessed for its biological activity in the EAE. system and its activity was comparable with that of linear agonist. EAE induced by cyclic analogue 2 was completely suppressed by the co-injection of the Ala⁸¹MBP₇₂₋₈₅ antagonist analogue. The comparable potencies of linear and cyclic analogues, indicate that the encephalitogenic linear peptide participates in the trimolecular complex with a cyclic conformation in which the carboxyl group of Asp at position 81 together with the guanidino group of Arg residue may play an important role for activation of this complex. Replacement of Asp by Ala results in disruption of interaction and antagonist activity.

A2. Conformational Model of Human MBP₈₇₋₉₉

Design of the cyclic analogues of Human MBP epitope 87-99 was based on Nuclear Magnetic Resonance and Molecular Dynamics. These studies revealed a head to tail intramolecular proximity (ROE connectivity between NHArg97-αVal87) indicating a pseudocyclic conformation for the immunogenic peptide MBP₈₇₋₉₉. Similarly NMR studies of [Arg⁹¹, Ala⁹⁶) MBP₈₇₋₉₉ (antagonist) revealed a head to tail intramolecular interaction (ROE connectivity between αPhe89-δ₂Pro99).

B. Advantages of Cyclic Analogues Over Linear

Cyclization of amino acid sequences results in increased metabolic stability, potency, receptor selectivity and bioavailability all of them reflecting a better pharmacological profile [Scott, et al 1999, Oligino, et al 1997]. In particular cyclic peptides have been used in several cases as synthetic immunogens [Bruggle, et al 1999], potent vaccine for diabetes [Berezhkovskiy, et al 1999], antigens for Herpes Simplex Virus [Mezo, et al 1999], transmembrane ion channels [Chaloin et al 1999], inhibitors of mHV-1 Tat-TAR interactions in human cells [Tamilarasu, 2000], of α-amylase, pancreatic tripsin and as protein stabilizer [Iwai, et al 1999].

Furthermore, advantages of cyclic analogues over their linear counterpart include (i) The cyclic analogues are more stable molecules and thus more resistant to enzymatic degradation, a quality that makes them attractive candidates as drug leads. (ii) It is an intermediate step towards the rational design and development of a non-peptide drug for oral administration, which is the ultimate goal of this work and technology. (iii) The conformation of the cyclic analogues is fixed compared to the conformational flexibility characterizing the linear counterparts. The active conformation of the potent linear peptides, which are very important for further drug development, has been identified through combined SAR, NMR and Dynamics studies.

To our knowledge, this is the first time that cyclic analogues of MBP epitopes have been synthesized and shown to prevent the development of EAE.

B1. Cyclic Analogues of Human MBP8799 Epitope Inducing and Suppressing the Developmeat of EAE

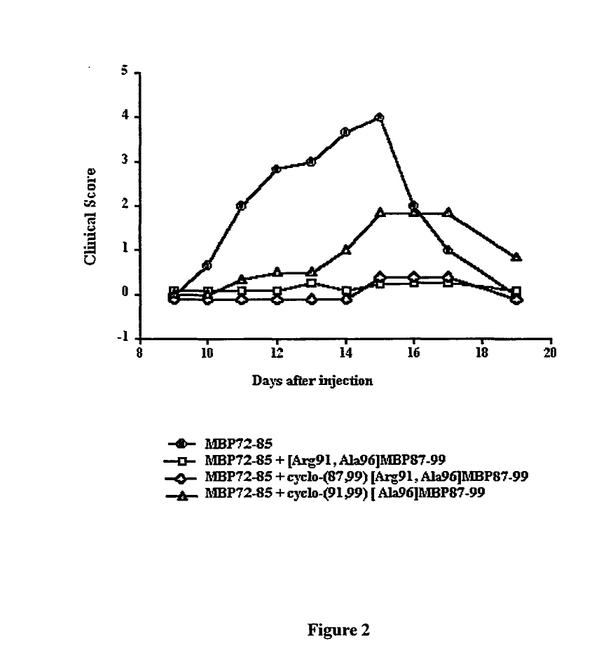

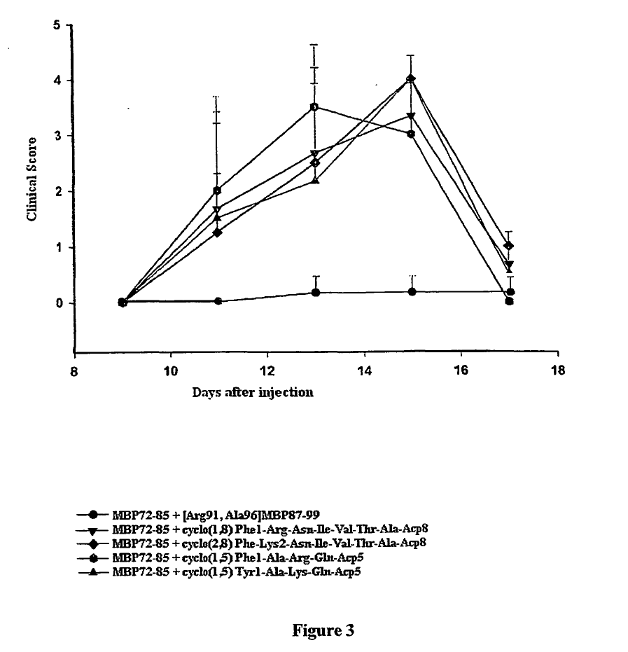

The MBP₇₂₋₈₅ peptide (25 μg) induced an acute monophasic disease with a peak clinical score at day 13 after the initial injection, followed by complete recovery in all animals by day 18. Coinjection of [Arg⁹¹, Ala⁹⁶]MBP₈₇₋₉₉ (500 μg) with the potent agonist peptide MBP₇₂₋₈₅ (25 μg) completely prevented the development of EAE. (FIG. 2) demonstrating that this linear antagonist is a potent mhibitor of disease induced by linear analogue MBP₇₂₋₈₅. Further modification and cyclization resulted in two cyclic antagonist peptides. The Lys side chain and C-terminus amide-linked cyclic analogue, cyclo-(91, 99) [Ala⁹⁶] MBP₈₇₋₉₉ (Val-His-Phe-Phe-Lys⁹¹-Asn-Ile-Val-Thr-Ala⁹⁶-Arg-Thr-Pro⁹⁹) had low inhibitory activity in the EAE system while the amide-linked cyclic analogue, cyclo-(87, 99) [Arg⁹¹, Ala⁹⁶] MBP₈₇₋₉₉ was a strong inhibitor of EAE when co-administered with MBP₇₂₋₈₅ (FIG. 2). Co-injection of any cyclic analogue (500 μg) with MBP₇₂₋₈₅ (25 μg) did not inhibit EAE activity. However all cyclic analogues shown in FIG. 3 except cyclo-(1, 5) Phe¹-Ala-Arg-Gln-Acp⁵ resulted in delay of onset of EAE (from day 13 to day 15) but not its severity (FIG. 3).

B2. Cyclic Analogue c-Ala⁸¹MBP₇₂₋₈₅ of Guinea Pig MBP 72-85 Epitope Potently Inhibits MBP₇₂₋₈₅ Induced EAE in Lewis Rats

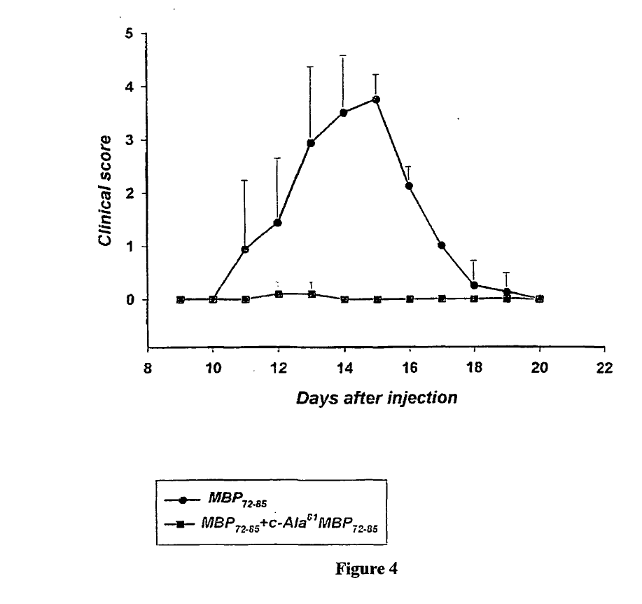

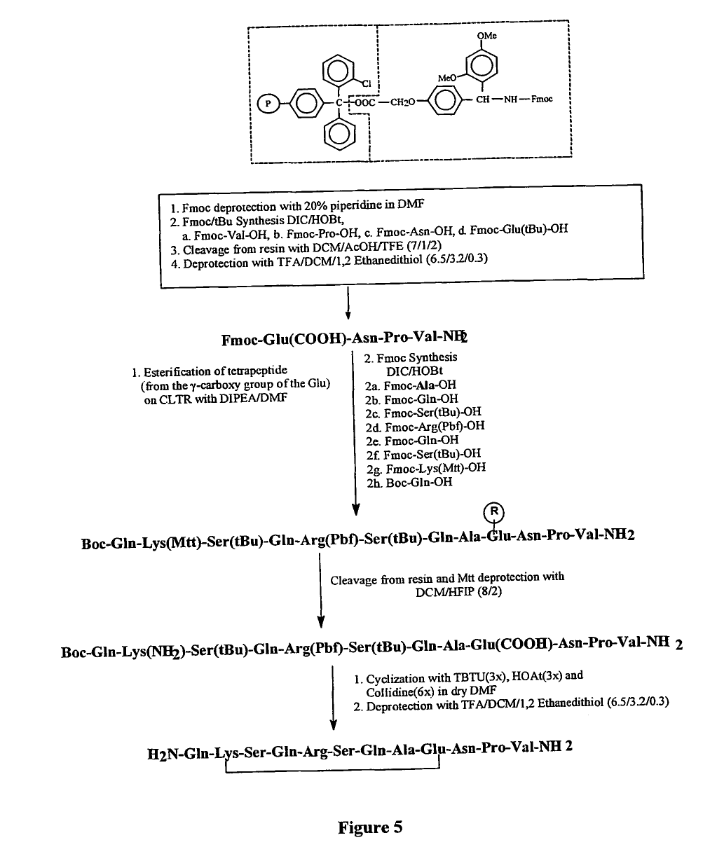

Analogues MBP₇₂₋₈₅ and c-MBP₇₂₋₈₅ induce EAE when injected subcutaneously in Lewis rats. Both these analogues induce an acute monophasic disease with a peak clinical score at day 15 following the initial injection, and eventual complete recovery in all animals. In the present study, MBP₇₂₋₈₅ was used to induce EAE and to evaluate the activity of the cyclic antagonist. When c-Ala⁸¹MBP₇₂₋₈₅ was co-injected with MBP₇₂₋₈₅ the clinical signs of EAE were completely prevented (FIG. 4), demonstrating that c-Ala⁸¹MBP₇₂₋₈₅ is a powerfill antagonist of MBP₇₂₋₈₅-induced EAE in Lewis rats. The result was confirmed at the histological level. Histopathological examination of spinal cord sections taken from MBP₇₂₋₈₅-injected animals, which were sacrificed at the peak of the disease, showed extensive perivascular and parenchymal inflammation throughout the length of the spinal cord as well as demyelination demonstrated by focal loss of luxol fast blue-stained myelin. In contrast, spinal cord section taken from rats immunized with MBP₇₂₋₈₅ and Ala⁸¹MBP₇₂₋₈₅ showed the complete absence of inflammation and demyelination.

C. Mannan/KLH Conjugates of MBP Epitopes (Linear or Cyclic/Agonists or Antagonists) in the Immunotherapy of EAE in Rats: Implications for its Use in MS

Mannan has been used as a successful carrier to target peptides to the macrophage/dentritic cell mannose receptor. Upon binding, MHC class I or MHC class II presentation of peptides is generated; stimulating either CTL/A6 or Th1/m2 immune responses. Preliminary results suggest that conjugations of reduced mannan to cyclic antagonist/agonist peptides are more potent than cyclic analogues alone. Further investigations are being done to measure cytokines and T cells after immunization of oxidized/reduced mannan conjugates to cyclic MBP analogues.

### DETAILED DESCRIPTION OF WORK

Synthesis of Guinea Pig MBP Cyclic Peptides

Cyclo-(2, 9)MBP₇₂₋₈₅: GIn-Lys²-Ser-Gln-Arg-Ser-Gln-Asp⁸¹-Glu⁹-Asn-Pro-Val-NH₂ Cyclo-(2, 9) Ala⁸¹MBP₇₂₋₈₅: Gln-Lys²-Ser-Gln-Arg-Ser-Gln-Ala⁸¹-Glu⁹-Asn-Pro-Val-NH₂

The synthesis of Fmoc-Glu(COOH)-Asn-Pro-Val-NH₂ was performed starting with H₂N-Linker(Rink)-2-chlorotrityl chloride resin. The Fmoc/tBu strategy and a single coupling protocol with N,N Diisopropylcarbodiimid/1-Hydroxybenzotriazole (DIC/HOBt) in dimethylformamide (DMF) was used through all syntheses. The amino acids were: Fmoc-Val-OH, Fmoc-Pro-OH, Fmoc-Asn-OH and Fmoc-Glu(tBu)-OH. The protected peptide on the resin was treated with the splitting mixture dichloromethane/acetic acid/2,2,2 trifluoroethanol (DCM/AcOH/TFE) (50 ml, 7:1:2) for 1 h at room temperature to remove the peptide from the resin. The mixture was filtered and the resin washed with the splitting mixture (X2) and DCM (X3). The solvent was removed on a rotary evaporator and the obtained oily product precipitated from dry diethyl ether as a white solid. The protected peptide-linker material was treated with 65% trifluroacetic acid (TFA)+3% ethanedithiol (EDT) in DCM for 4 h to deprotect Glu from tBu and to liberate the amidated tetrapeptide fragment from linker. 2-Chlorotrityl chloride resin in dry DMF was stirred in a round bottom flask. Diisopropylethylamine (DIPEA) and Fmoc-Glu(COOH)-Asn-Pro-Val-NH₂ added and the solution was stirred for 45 min at room temperature. A mixture of methanol (MeOH) and DIPEA (8/2) was then added for endcaping and the resulting Tnmxture was stirred for another 10 min at room temperature. The Fmoc-Glu(Resin)-Asn-Pro-Val-NH₂ was used for the synthesis of the linear precursor peptide following the protocol previously described [Tselios, et al 1999]. The amino acids used in Fmoc synthesis were: Fmoc-Ala-OH, FmocAsp(tBu)-OH, Fmoc-Gln-OH, Fmoc-Ser(tBu)-OH, Fmoc-Arg(Pbf)-OH, Fmoc-Lys(Mtt)-OH and the last amino acid was Boc-Gln-OH. The completed peptide on resin was dried in vacuo and then treated with the splitting mixture DCMI1,1,1,3,3,3 hexafluoro-2-propanol (8/2) for 6 h at room temperature to remove the peptide from the resin and for the deprotection of Lys from Mtt. The mixture was filtered and the resin was washed with the splitting mixture (X2) and DCM (X3). The solvent was removed on a rotary evaporator and the obtained oily product precipitated from cold dry diethyl ether as a white solid. Cyclization of linear protected peptide was achieved using O-benzotriazol-1-yl-N,N,N′,N′-tetramethyluronium tetrafluoroborate (TBTU), 2,4,6 collidine, 1 hydroxy 7-azabenzotriazol and dry DMF as solvent. The solution of peptide, 2,4,6 collidine and 1 hydroxy 7-azabenzotriazol in dry DMF was added dropwise to a solution of TBTU in DMF for 2 h and the solution was stirred for 3 h. The protected cyclic peptide was treated with 65% TFA in DCM+3% EDT for 5 h at room temperature (FIG. 5). The crude peptide product was purified further by preparative HPLC.

Synthesis of Human MBP Cyclic Peptides

Cyclo-(87, 99) [Arg⁹¹, Ala⁹⁶] MBP₈₇₋₉₉ (Val⁸⁷-His-Phe-Phe-Arg⁹¹-Asn-Ile-Val-Thr-Ala⁹⁶-Arg-Thr-Pro⁹⁹)

Cyclo-(91, 99) [Ala⁹⁶] MBP₈₇₋₉₉ (Val⁸⁷-His-Phe-Phe-Lys⁹¹-Asn-Ile-Val-Thr-Ala⁹⁶-Arg-Thr-Pro⁹⁹)

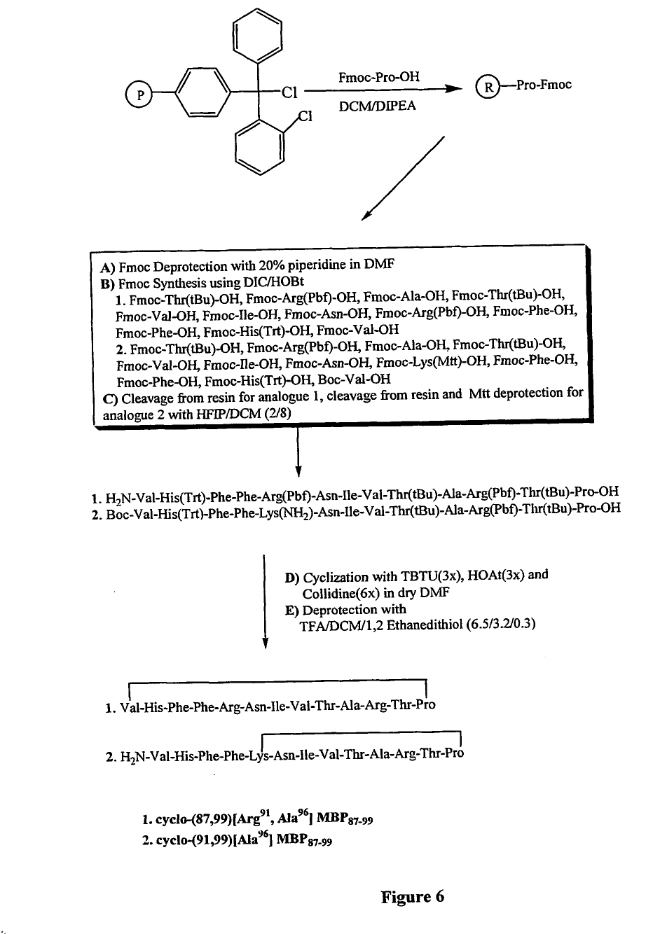

For the synthesis of precyclic human MBP cyclic analogues, we resorted to the Fmoc/tBu methodology utilizing the 2 chlorotrityl chloride resin. The peptide synthesis was achieved using DIC/HOBt in DMF and the Na-NH₂ of amino acids were protected with the Fmoc group. The side chain of amino acids were protected as following: Trt for His, Pbf for Arg, tBu for Ser, Thr, Asp, Glu, Boc for Lys, as regarding the cyclic analogue cyclo-(91, 99)[Ma⁹⁶] MBP₈₇₋₉₉ (by NE-NH₂ of Lys and C-terminous) we used Mtt protected: group because it easily removed with the mixture HFIP(1,1,1,3,3,3]hexafluoro-2-propanol)/DCM (8/2) in which the peptide cleavages from the resin. Otherwise, in the cyclo-(87, 99)[Arg⁹¹, Ala⁹⁶]MBP₈₇₋₉₉ the side chain of Lys was protected with Boc group. The completed protected linear peptides on resin were dried in vacuo and then treated with the splitting mixture DCM/HFIP (8/2) for 7 h at room temperature to remove the peptide from the resin and for the deprotection of Lys from Mtt in the cyclo-(91, 99)[Ala⁹⁶] MBP₈₇₋₉₉. Each one of the linear protected peptides was dissolved in DMF and was added collidine, HOAt. This mixture was added dropwise in a solution of TBTU in DMF for 8 hours. The cyclization was determined by TLC and analytical reversed phase HPLC. The solvent was removed under reduced pressure affording a light yellow oily residue. The cyclic protected peptide (purity ≧90%) precipitated from H₂O and dried in vacuo for 16 h. The cyclic protected peptide was treated with 65% TFA in DCM and 3% ethanodithiol as scavanger for 4 hours at room temperature. The resulting solution was concentrated to small volume and the final free peptide was precipitated as a light yellow amorphous solid added diethylether (purity ≧80%). Peptide purity was assessed by analytical HPLC reruns, thin layer chromatography (TLC) and mass spectrometry (ESIMS) (FIG. 6).

Conjugation of Peptide to Mannan and/or KLH

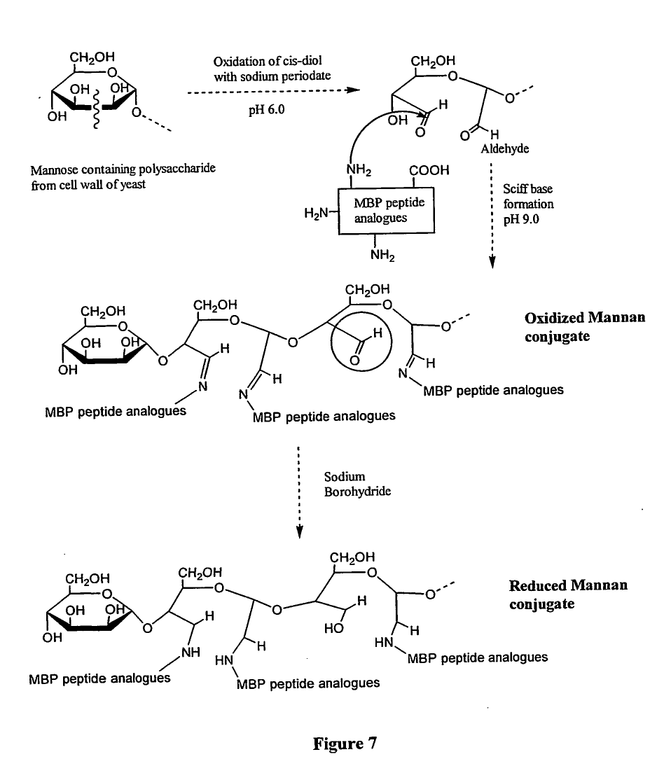

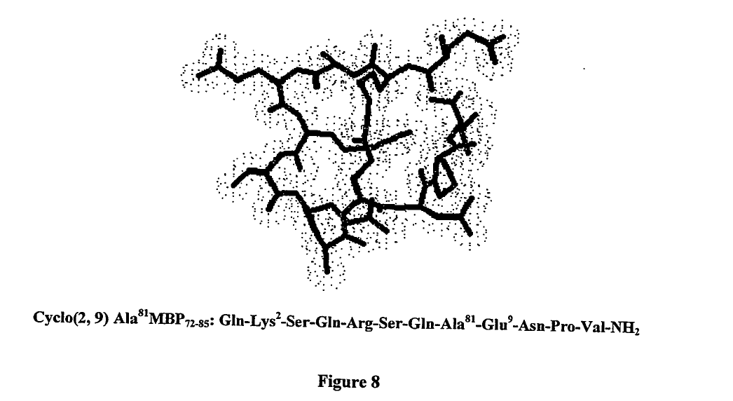

1 mg/ml mannan (0.1M phosphate buffer pH 6.0) is oxidized to a poly-aldehyde by treating with 100 μl 0.1M sodium periodate, for 1 hour at 4° C. 10 μl ethanedithiol is added for 30 minutes at 4° C. to stop oxidation. The mixture is passed through a PD-10 column and the mannan fraction is collected. The PD-10 column pre-calibrated with 0.1M carbonate buffer pH 9.0. The void volume of the PD-10 column is 2.5 ml, the oxidized mannan (1 ml) is added, then is added 1.5 ml 0.1M carbonate buffer pH 9.0. The following 2 ml are collected. For the conjugation to mannan, 1 mg of each peptide [Linked to KLH or Lys residues added (Lys-Gly-Lys-Gly-Lys-Gly-Lys-Gly-Lys-Gly)] is allowed to react with oxidized mannan overnight at room temperature. The antigen will be conjugated to mannan in the oxidized form (contains aldehydes and Schiff bases) (Scheme 2). For conjugation of antigen to mannan in the reduced form (reducing the aldehydes to alcohol's and Schiff baces to amines): Oxidized mannan antigen complex is reacted with 1 mg sodium borohydride for 3 hours at room temperature. The conjugation is used with no further purification (FIG. 7).

Induction-Inhibition and Assessment of EAE

Inbred Lewis rats bred and maintained in the animal facility of the Hellenic Pasteur Institute were used in all experiments. Female rats (220g) were immunised with linear MBP₇₂₋₈₅ (30 μg) (n=10, as positive control), or MBP₇₂₋₈₅ (30 kg) plus the cyclic analogue c-Ala⁸¹MBP₇₂₋₈₅ (500 μg) or [Arg⁹¹,Ala⁹⁶] MBP₈₇₋₉₉ (n=5) in 200 μl of an emulsion containing equal volumes of peptide diluted in sterile saline and Freund's complete adjuvant (Difco, USA) containing 4 mg/ml heat-killed M. tuberculosis (H37Ra) (Difco). Immunisation was performed subcutaneously in the two hind foot pads and repeated 7 days later with the same dosage. Rats were examined daily for clinical signs of EAE and scored as following: 0, no clinical disease; 0.5, weight loss; 1, tail weakness; 2, paraparesis of hindlimbs; 3, paraplegia of hindlimbs; 4, paraplegia with forelimb weakness, moribund; 5, death. PBS/CFA-injected arirnals served as negative controls. For histological analyses, mice were anaesthetised with ether, bled and perfused with PBS (pH 7.4) (PBS) followed by 4% paraformaldehyde in PBS. Spinal cord was dissected out and fixed overnight in 4% paraformaldehyde in PBS at 4° C. before been embedded in paraffin. Paratfin sections were stained with Luxol fast blue and Nuclear Fast Red for visualisation of demyelination and inflammation respectively.

What is claimed:

1. A conformational model of linear epitope sequence from guinea pig MBP₇₂₋₈₅ and human MBP₈₇₋₉₉.

2. The cyclic agonist(cyclo-MBP₇₂₋₈₅) and cyclic antagonist(cyclo-Ala⁸¹MBP₇₂₋₈₅) peptide derived from guinea pig MBP epitope 72-85 for the treatment of Multiple Sclerosis (MS).

3. The antagonist linear peptides [X⁹¹, Y⁹⁶]MBP₈₇₋₉₉ derived from human MBP epitope 87-99 where X=Arg, Lys, Asn, Ala, Orn and Y=Ala, Gly, Val, Pro for the treatment of Multiple Sclerosis (MS).

4. The antagonist cyclic peptides cyclo(87-99) [X⁹¹, Y⁹⁶]MBP₈₇₋₉₉, cyclo(91-96) [X⁹¹, Y⁹⁶]MBP₈₇₋₉₉ derived from human MBP epitope 87-99 where X, Y as in claim 3 for the treatment of Multiple Sclerosis (MS).

5. The Mannan/KLH conjugates with linear MBP₇₂₋₈₅ agonist, linear/cyclic Ala⁸¹MBP₇₂₋₈₅ antagonists and linear/cyclic [X⁹¹, Y⁹⁶]MBP₈₇₋₉₉ antagonists in the oxidised and reduced form of mannan where X, Y as in claim 3, 4 for the treatment of Multiple Sclerosis (MS).

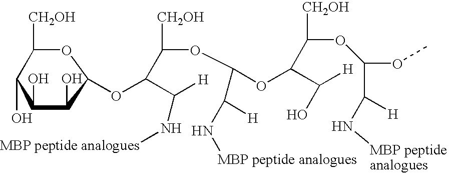

6. A method for the synthesis of guinea pig cyclic analogues as in claim 2.

7. A method for the synthesis of human cyclic analogues as in claim 4.

8. A method for the synthesis of peptides-KLH/Mannan conjugates as in claim 5.
